(19) United States
(12) Patent Application Publication
(10) Pub. No.: US 2023/0010168 A1
(43) Pub. Date: Jan. 12, 2023

(54) WEARABLE ELECTRONIC DEVICE FOR DETECTING BIOMETRIC INFORMATION

(71) Applicant: Samsung Electronics Co., Ltd., Gyeonggi-do (KR)

(72) Inventors: Hyunjun JUNG, Gyeonggi-do (KR); Jinho KIM, Gyeonggi-do (KR)

(21) Appl. No.: 17/939,148

(22) Filed: Sep. 7, 2022

Related U.S. Application Data
(63) Continuation of application No. PCT/KR2021/003283, filed on Mar. 17, 2021.

(30) Foreign Application Priority Data
Mar. 18, 2020 (KR) 10-2020-0033158

(51) Int. Cl. A61B 5/00; A61B 5/282; A61B 5/0531

(52) U.S. Cl. CPC A61B 5/6843; A61B 5/282; A61B 5/0531; A61B 5/7203; A61B 5/681; A61B 2560/0443; A61B 2562/0209; A61B 5/0059; A61B 2562/046

(57) ABSTRACT

According to certain embodiments, a wearable electronic device comprises: a housing; a first electrode and a second electrode disposed on the housing; an A/D converter connected to the first electrode and the second electrode; a pulse generator connected to the first electrode and the second electrode; and a processor operatively connected to the A/D converter and the pulse generator; wherein the processor is configured to: control the pulse generator to output a series of pulse waves to the first electrode when an external object is in contact with the first electrode; and controlling the A/D converter to obtain biometric information from the first electrode and second electrode in response to outputting the series of pulse waves.

### CLAIM OF PRIORITY

This application is a continuation of International Application No. PCT/KR2021/003283 filed Mar. 17, 2021, that, in turn, claims priority to Korean Patent Application No. 10-2020-0033158 filed in the Korean Intellectual Property Office on Mar. 18, 2020.

### BACKGROUND

### 1. Technical Field

Certain embodiments disclosed in the disclosure relate to a wearable electronic device for sensing biometric information.

### 2. Background Art

Recently, wearable electronic devices are becoming widespread. Accordingly, the various functions that are implemented in wearable electronic devices is increasing.

Due to direct contact with the human, it may be possible to provide health care service through wearable electronic devices. In order to provide a health care services, methods for obtaining accurate biometric information are needed.

Accuracy of detected biometric signal may be reduced due to the user's wearing environment and/or wearing state.

Certain embodiments may provide a wearable electronic device capable of reducing resistance and contact impedance of a skin in contact with an electrode. The foregoing may minimize signal interference due to noise. As a result, the wearable electronic device provide more accurately measures biometric information.

### SUMMARY

According to certain embodiments, a wearable electronic device comprises: a housing; a first electrode and a second electrode disposed on the housing; an A/D converter connected to the first electrode and the second electrode; a pulse generator connected to the first electrode and the second electrode; and a processor operatively connected to the A/D converter and the pulse generator; wherein the processor is configured to: control the pulse generator to output a series of pulse waves to the first electrode when an external object is in contact with the first electrode; and controlling the A/D converter to obtain biometric information from the first electrode and second electrode in response to outputting the series of pulse waves.

According to certain embodiments, a method comprises: outputting a series of pulse waves to a first electrode when an external object is in contact with the first electrode; and obtaining biometric information by an A/D converter from the first electrode and a second electrode in response to outputting the series of pulse waves.

### BRIEF DESCRIPTION OF THE DRAWINGS

FIG. 1 is a block diagram illustrating an electronic device in a network environment according to certain embodiments.

FIG. 2 is a plan view of a wearable electronic device according to one embodiment.

FIG. 3 is a block diagram of a wearable electronic device according to one embodiment.

FIG. 4 is an operation flowchart of a wearable electronic device according to one embodiment.

FIG. 5 is a graph showing contact impedance over time of a wearable electronic device according to one embodiment.

FIG. 6 is a waveform diagram of a series of pulse waves output from a wearable electronic device according to one embodiment.

FIG. 7 is a block diagram of a wearable electronic device according to one embodiment.

FIG. 8 is an operation flowchart of a wearable electronic device according to one embodiment.

FIG. 9 is a graph showing contact impedance over time of a wearable electronic device according to one embodiment.

FIG. 10 is an operation flowchart of a wearable electronic device according to one embodiment.

FIG. 11 is a circuit diagram illustrating an A/D converter of a wearable electronic device according to one embodiment.

FIG. 12 is a circuit diagram showing an A/D converter of a wearable electronic device according to another embodiment.

In connection with the description of the drawings, the same or similar reference numerals may be used for the same or similar components.

### DETAILED DESCRIPTION

According to certain embodiments, the wearable electronic device may reduce the resistance and the contact impedance of the skin in contact with the electrode, thereby minimizing signal interference due to noise, and thus measuring accurate biometric information.

In certain embodiments, an electrode in contact with the user's skin may transmit a series of electronic pulses. The electronic pulses may temporarily create a micro-channel in a surface of a cell membrane of the user's skin. When the micro-channel is created, the mobility of electrons through the micro-channel may increase, thereby reducing skin resistance. The reduced skin resistance may allow for more accurate measurement of biometric information.

Hereinafter, certain embodiments of disclosure will be described with reference to the accompanying drawings. However, this is not intended to limit the disclosure to specific embodiments, and it should be understood that the disclosure includes various modifications, equivalents, and/or alternatives of the embodiments of the disclosure.

### Electronic Device

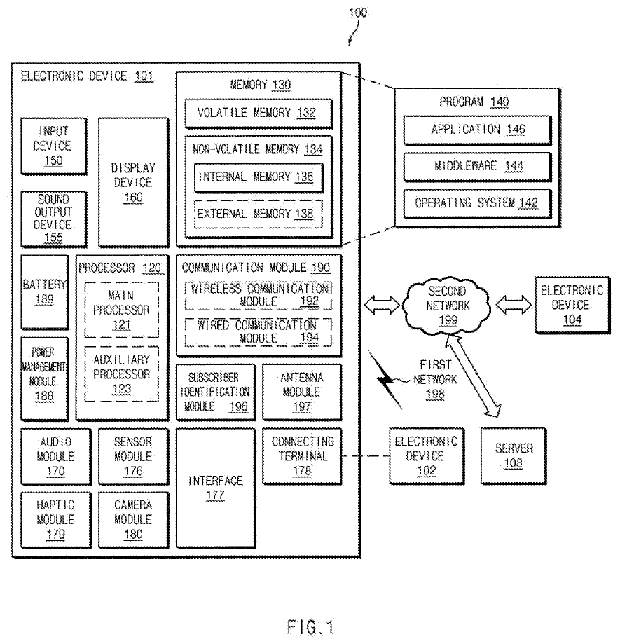

FIG. 1 is a block diagram illustrating an electronic device 101 in a network environment 100 according to certain embodiments. Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input device 150, a sound output device 155, a display device 160, an audio module 170, a sensor module 176, an interface 177, a haptic module 179, a camera module 180, a power management module 188, a battery 189, a communication module 190, a subscriber identification module (SIM) 196, or an antenna module 197. In some embodiments, at least one (e.g., the display device 160 or the camera module 180) of the components may be omitted from the electronic device 101, or one or more other components may be added in the electronic device 101. In some embodiments, some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may load a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The auxiliary processor 123 may be implemented as separate from, or as part of the main processor 121.

The term “processor” shall be understood to refer to both the singular and plural contexts in this document.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input device 150 may receive a command or data to be used by other component (e.g., the processor 120) of the electronic device 101, from the outside (e.g., a user) of the electronic device 101. The input device 150 may include, for example, a microphone, a mouse, a keyboard, or a digital pen (e.g., a stylus pen).

The sound output device 155 may output sound signals to the outside of the electronic device 101. The sound output device 155 may include, for example, a speaker or a receiver. The speaker may be used for general purposes, such as playing multimedia or playing record, and the receiver may be used for an incoming calls. According to an embodiment, the receiver may be implemented as separate from, or as part of the speaker.

The display device 160 may visually provide information to the outside (e.g., a user) of the electronic device 101. The display device 160 may include, for example, a display, a hologram device, or a projector and control circuitry to control a corresponding one of the display, hologram device, and projector. According to an embodiment, the display device 160 may include touch circuitry adapted to detect a touch, or sensor circuitry (e.g., a pressure sensor) adapted to measure the intensity of force incurred by the touch.

The audio module 170 may convert a sound into an electrical signal and vice versa. According to an embodiment, the audio module 170 may obtain the sound via the input device 150, or output the sound via the sound output device 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., PCB). According to an embodiment, the antenna module 197 may include a plurality of antennas. In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

Certain electronic devices 101 can be worn on the body. For example, an electronic device 101 can be equipped with wrist straps, thereby providing a smartwatch.

Additionally, due to direct contact with the user's skin, a wearable electronic device can measure biometric signals and provide various health related services. The wearable electronic device can include a plurality of electrodes that are configured to measure electrical signals from the user's body. However, electrical resistance and impedance between the electrodes may reduce the accuracy of the measured electrical signals.

Accordingly, in certain embodiments, an electrode transmits a series of electrical pulses. The electronic pulses may temporarily create a micro-channel in a surface of a cell membrane of the user's skin. When the micro-channel is created, the mobility of electrons through the micro-channel may increase, thereby reducing skin resistance. The reduced skin resistance may allow for more accurate measurement of biometric information.

### Wearable Electronic Device

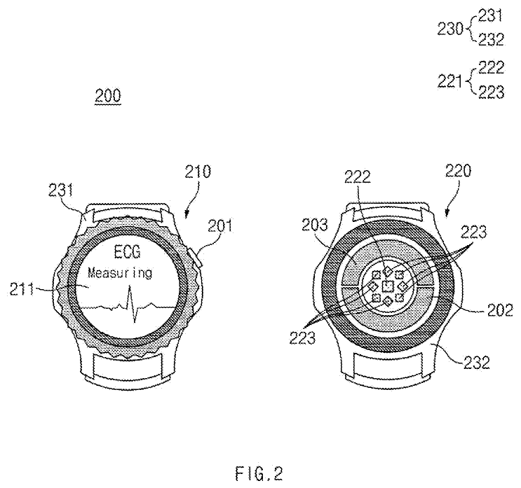

Hereinafter, with reference to FIG. 2, a wearable electronic device according to one embodiment will be described. FIG. 2 is a plan view showing a wearable electronic device 200 (e.g., the electronic device 101 in FIG. 1) according to one embodiment. Specifically, FIG. 2 shows a front surface 210 and a rear surface 220 of the wearable electronic device 200 according to one embodiment. In following embodiments, an operation of the wearable electronic device 200 may be referred to as an operation of a processor (e.g., the processor 120 in FIG. 1). The wearable electronic device 200 may include wrist straps to affix the wearable electronic device 200 to the user's wrist.

Referring to FIG. 2, the wearable electronic device 200 according to one embodiment may include a housing 230, a display 211 (e.g., the display device 160 of FIG. 1), an optical sensor 221 (e.g., the sensor module 176 in FIG. 1), and a plurality of electrodes 201, 202, or 203. Due to direct contact with the user's body, the electrodes 201, 202, or 203 can be positioned to take biometric measurements.

The housing 230 may include a front plate 231 and a rear plate 232 facing each other. The front plate 231 of the housing 230 may constitute a front surface (e.g., a third surface) of the housing, and the rear plate 232 thereof may constitute a rear surface (e.g., a second surface) opposite the front surface of the housing 230. Further, the housing 230 may include a side surface (e.g., a first surface) positioned between the front and rear surfaces so as to connect the front and rear surfaces to each other. The side surface of the housing 230 may include at least a portion of the front plate 231, or may include at least a portion of the rear plate 232. According to an embodiment, the side surface of the housing 230 may include a separate plate. The housing 230 may surround and protect components included in the wearable electronic device 200, or may fix some components thereof.

The display 211 may be exposed to an outside through a portion of the front plate 231. The display 211 may be configured to emit light having at least one wavelength and provide visual information to the user. Further, the display 211 may receive a user input (e.g., a touch input).

The optical sensor 221 may include a light source 222 emitting light and a plurality of light detectors 223 for detecting light.

The light source 222 may include at least one light emitting element (e.g., a light emitting diode (LED)) for irradiating light having a wavelength of a specified range. In one example, the light emitting elements may be configured to emit light of different wavelengths, respectively. In another example, at least some of the light emitting elements may be configured to emit light of the same wavelength. In still another example, the light emitting elements may emit light at the same time point or may emit light based on a specified pattern.

The plurality of light detectors 223 may detect light, and may detect an intensity of the detected light. In one example, the plurality of light detectors 223 may output a current signal having a magnitude corresponding to a detected amount of light. The plurality of light detectors 223 may be arranged to surround the light source 222. In FIG. 2, the plurality of light detectors 223 are shown as including 8 light detectors 223. However, the number and/or positions of the light detectors 223 are not limited thereto. The wearable electronic device 200 according to one embodiment may sense biometric information (e.g., heart rate, oxygen saturation, blood pressure, and/or blood sugar) associated with the user using the optical sensor 221. In one example, the optical sensor 221 may be a photoplethysmogram (PPG) sensor. The wearable electronic device 200 according to one embodiment may detect whether the user wears the wearable electronic device 200 using the optical sensor 221.

The optical sensor 221 may further include an optical signal processing module (not shown) electrically connected to the light source 222 and the plurality of light detectors 223. The optical signal processing module may acquire and process a current signal generated by the plurality of light detectors 223 based on the detected amount of light. The optical signal processing module may detect the user's biometric information or detect whether the user wears the wearable electronic device 200, based on an electrical signal obtained via the plurality of light detectors 223.

The plurality of electrodes 201, 202, or 203 may include the first electrode 201 positioned on the side surface (e.g., the first face) of housing 230, the second electrode 202 and the third electrode 203 positioned on the rear surface (e.g., the second surface) of the housing 230. However, the positions of the plurality of electrodes 201, 202, or 203 are not limited thereto. The electrodes may be positioned on another face of the housing 230. The first electrode 201 and the second electrode 202 may collect biometric signals at different positions, respectively. The third electrode 203 may be used for in-phase component noise reduction and biometric bias. In one example, the in-phase component noise may mean a noise signal (e.g., a power noise) input commonly to the first electrode 201 and the second electrode 202.

However, resistance and contact impedance can reduce the accuracy of biometric signals collected by the first electrode 201, second electrode 202, and third electrode 203. Accordingly, certain embodiments may reduce the resistance and the contact impedance of the skin in contact with the first electrode 201, second electrode 202, and third electrode 203. This reduces, if not minimizes signal interference due to noise. As a result, accuracy of biometric information measurements is improved.

The A/D converter of the wearable electronic device 200 according to one embodiment may obtain a biometric signal via a differential amplification circuit that rejects a common input and amplifies a differential input to obtain an output signal. The differential amplification circuit may reject the common input based on a common mode rejection ratio (CMRR) indicating a ratio of a common gain and a differential gain. The wearable electronic device 200 according to one embodiment may output a signal equal to the in-phase component noise input to the first electrode 201 and the second electrode 202 through the third electrode 203 to cancel the in-phase component noise, such that common input rejection performance of the differential amplification circuit may be improved.

When the user wears the wearable electronic device 200 according to one embodiment, the second electrode 202 and the third electrode 203 may touch one arm of the user. When the user wants to measure biometric information thereof, the user may touch a finger of a hand of the other arm to the first electrode 201. In another example, when the user does not wear the wearable electronic device 200, the second electrode 202 and the third electrode 203 may touch a palm or some of fingers of one hand of the user, and the first electrode 201 may touch fingers of the other hand. In the wearable electronic device 200, one of the first electrode 201, the second electrode 202, and the third electrode 203 may be spaced apart from the user's skin, and the remaining two electrodes come into contact with the user's skin to measure biometric information (e.g., galvanic skin response, electromyography).

In the wearable electronic device 200 according to one embodiment, in a state in which an external object (e.g., the user's skin) is in contact with at least one of the first electrode 201, the second electrode 202, and/or the third electrode 203 (the at least one electrode), and the at least one electrode may output a series of pulse waves. The at least one electrode may transmit the series of pulse waves as output an external object (e.g., the user's skin) in contact, therewith.

The wearable electronic device 200 according to one embodiment may collect a biometric signal via the plurality of electrodes 201, 202, or 203 in contact with the user's skin. In this regard, impedance (hereinafter, referred to as contact impedance) may be generated at an area in which the plurality of electrodes 201, 202, or 203 and the user's skin contact each other. The contact impedance may be determined based on an external environment (e.g., temperature or humidity), a wearing state (e.g., a contact area or a contact site) and/or the user's skin type. Therefore, when the biometric signal is collected using an electrical signal, the contact impedance may affect quality of the collected biometric signal. In one example, as the contact impedance is changed, a noise level may increase or accuracy of the biometric signal may be deteriorated.

The wearable electronic device 200 according to one embodiment may lower the contact impedance using electroporation. The electroporation refers to a scheme to apply an electric field to a cell to increase permeability of a plasma membrane. According to the electroporation, an electric pulse may be applied to the skin to temporarily create a micro-channel in a surface of a cell membrane. When the micro-channel is created in the surface of the cell membrane, mobility of electrons through the micro-channel may increase, thereby reducing skin resistance. The wearable electronic device 200 according to one embodiment may output an electrical pulse through at least one of the plurality of electrodes 201, 202, or 203 in contact with the user's skin, thereby reducing the skin resistance. As a result, the contact impedance between the user's skin to which the electric pulse is applied and the plurality of electrodes 201, 202, or 203 may be reduced.

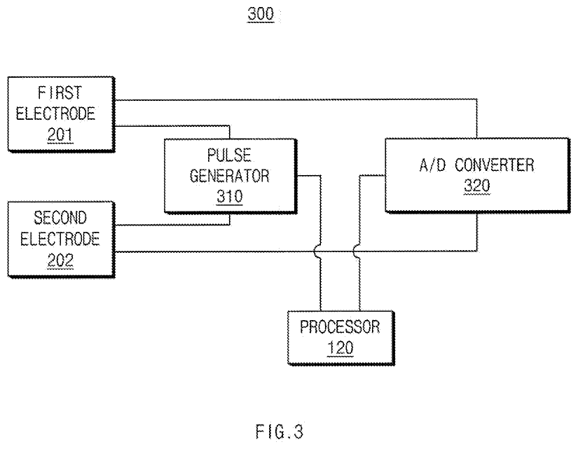

Hereinafter, with reference to FIG. 3, a wearable electronic device according to one embodiment will be described. FIG. 3 is a block diagram 300 of a wearable electronic device according to one embodiment (e.g., the wearable electronic device 200 in FIG. 2).

Referring to FIG. 3, the wearable electronic device 200 according to one embodiment may include the processor 120, the first electrode 201, the second electrode 202, a pulse generator 310, and/or an A/D converter 320. In following embodiments, an operation of the wearable electronic device 200 may be referred to as an operation of the processor 120.

The pulse generator 310 may be electrically connected to the first electrode 201 and/or the second electrode 202 so as to output a series of pulse waves to at least one of the first electrode 201 and/or the second electrode 202. The pulse generator 310 may receive specified pulse information and output the series of pulse waves based on the pulse information. In one example, the pulse generator 310 may include an LC resonant circuit that generates the series of pulse waves. According to one embodiment, the wearable electronic device 200 may further include a switch (not shown) connecting the pulse generator 310 and at least one of the first electrode 201 and the second electrode 202 to each other.

The A/D converter 320 may be electrically connected to the first electrode 201 and the second electrode 202 so as to detect biometric information (e.g., electrocardiogram (ECG), bioelectrical impedance analysis (BIA), and/or electrodermal activity (EDA)). The A/D converter 320 may process an electrical signal obtained via the first electrode 201 and the second electrode 202. In one example, the A/D converter 320 may process (e.g., amplify and/or filter) the electrical signal measured by the first electrode 201 and the second electrode 202, and may convert the processed electrical signal into a digital signal.

Although only the first electrode 201 and the second electrode 202 are shown in FIG. 3, the number of electrodes is not limited thereto. The wearable electronic device 200 according to one embodiment may include three or more electrodes. In one example, the wearable electronic device 200 according to one embodiment may further include the third electrode 203 (refer to FIG. 2) electrically connected to the pulse generator 310 and the A/D converter 320.

The processor 120 may be electrically or operatively coupled or connected to other components (e.g., the pulse generator 310 and the A/D converter 320) of the wearable electronic device 200 and may be configured to control the other components of the wearable electronic device 200.

The processor 120 may determine whether the first electrode 201 and the second electrode 202 are in contact with an external object (e.g., the user's skin). In the wearable electronic device 200 according to one embodiment, the processor 120 may determine whether the first electrode 201 and the second electrode 202 are in contact with the external object. The processor 120 may transmit a voltage from a voltage source. The processor 120 may determine contact based on an output value of a contact detection module (e.g., a comparator) in response to transmitting the voltage.

In another example (not shown), the processor 120 may be electrically connected to the first electrode 201 and the second electrode 202, and may detect a change in capacitance between the first electrode 201 and the second electrode 202 and thus determine whether the first electrode 201 and the second electrode 202 are in contact with the external object, based on the detected change. According to one embodiment, the A/D converter 320 of the wearable electronic device 200 may determine whether the first electrode 201 and/or the second electrode 202 is in contact with the external object, using the first electrode 201 and the second electrode 202. In this case, the A/D converter 320 may transmit information regarding whether the first electrode 201 and the second electrode 202 are in contact with the external object to the processor 120.

In another example, the processor 120 may determine whether the second electrode 202 is in contact with the external object, using the optical sensor (221 in FIG. 2). In one embodiment, the processor 120 may determine whether the second electrode 202 is in contact with the external object, based on an amount of light detected by the light detector (223 in FIG. 2) of the optical sensor 221. In one example, the processor 120 may determine that the second electrode 202 is positioned within a specified distance from the external object (or is in contact with the external object) in an area in which an amount of light equal to or greater than a specified level is detected. In another example, when the amount of light detected by the light detector (223 in FIG. 2) of the optical sensor 221 is lower than the specified level, the processor 120 may determine that the second electrode 202 does not contact the external object. When the wearable electronic device 200 according to one embodiment includes the second electrode 202 and the third electrode 203 (see FIG. 2) on the rear surface of the housing (230 in FIG. 2), the processor 120 may determine whether the second electrode 202 and the third electrode 203 are in contact with the external object using the optical sensor (221 in FIG. 2).

In one embodiment, the wearable electronic 200 may include mechanical pressure sensors. When at least one of the first electrode 201, second electrode 202, and the third electrode 203 make contact with a user, the at least one electrode may be pressed in a direction. The pressure sensor may provide a notification to the processor 120.

The processor 120 may determine pulse information for the series of pulse waves. The pulse information for the series of pulse waves may include at least one parameter about the series of pulse waves. In one example, the parameter included in the pulse information may include at least one of pulse power, a pulse width, a pulse interval, a pulse period, a pulse train width, a pulse train interval, pulse amplitude, a pulse train period, the number of pulses included in each pulse train, a duty cycle, and/or a pulse shape.

The processor 120 may be configured to output the series of pulse waves according to pulse information through at least one of the first electrode 201 and/or the second electrode 202 using the pulse generator 310. That is, when the first electrode 201 and/or the second electrode 202 are detected to be in contact with an external object, the processor 310 can cause the pulse generator 310 to transmit a pulse to one of the first electrode 201 and/or the second electrode 202

The processor 120 may obtain biometric information using the A/D converter 320, the first electrode 201, and the second electrode 202. The processor 120 may perform post-processing (e.g., filtering and/or noise canceling) on the biometric information detected by the A/D converter 320.

According to certain embodiments, the wearable electronic device 200 sets target impedance and pulse information and waits until the first electrode 201 and second electrode 202 make contact with an object, such as the user's skin. After determining that the first electrode 201 and second electrode 202 have made contact, the pulse generator 310 transmits a series of pulse waves and measures the user's biometric information.

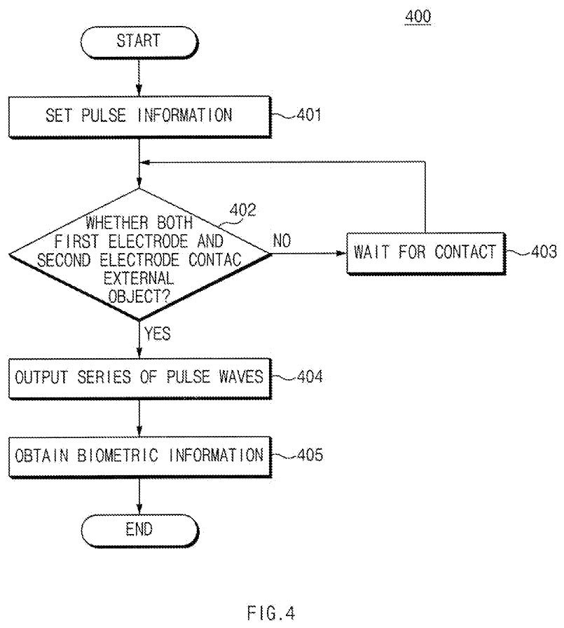

Hereinafter, with reference to FIG. 4, an operation of the wearable electronic device according to one embodiment (e.g., the wearable electronic device 200 in FIG. 2) will be described. FIG. 4 is an operation flow diagram 400 of the wearable electronic device 200 according to one embodiment. In following embodiments, an operation of the wearable electronic device 200 may be referred to as an operation of the processor 120.

In operation 401, the wearable electronic device 200 according to one embodiment may set pulse information. The pulse information may include at least one parameter about the series of pulse waves. In one example, the wearable electronic device 200 according to one embodiment may set at least one parameter value about the series of pulse waves. In another example, the wearable electronic device 200 according to one embodiment may select at least one pulse information among the pulse information stored in a memory (e.g., the memory 130 in FIG. 1).

In operation 402, the wearable electronic device 200 according to one embodiment may determine whether the first electrode (201 in FIG. 3) and the second electrode (202 in FIG. 3) contact the external object (e.g., the user's skin). In one example, the wearable electronic device 200 may determine whether the first electrode 201 and the second electrode 202 are in contact with the external object. The foregoing can be determined, based on the output value of the contact detection module (e.g., the comparator). Voltage can be transmitted by a voltage source. Contact with an external object can be determined based on the detected change in capacitance between the first electrode 201 the second electrode 202. In another example, the wearable electronic device 200 according to one embodiment may determine whether the second electrode 202 is in contact with the external object using the optical sensor (221 in FIG. 2). It is noted that the wearable electronic device 200 is not limited to determining contact by the foregoing means, and other means may also be used.

When it is determined that the first electrode 201 and the second electrode 202 are not in contact with the external object, the wearable electronic device 200 may wait for contact in operation 403. In one example, the wearable electronic device 200 according to one embodiment may simply wait for a specified time duration (polling). In another example, the wearable electronic device 200 according to one embodiment may output a re-wearing request message and/or a re-contacting message to the user via a display (e.g., the display 211 in FIG. 2) when the user wants to measure the biometric information (e.g., execute a measuring application or periodically set measurement). After the wearable electronic device 200 according to one embodiment has performed the contact waiting operation (operation 403), the wearable electronic device 200 may re-determine whether the first electrode 201 and the second electrode 202 are in contact with the external object (in operation 402). In one example, the wearable electronic device 200 may periodically perform operation 402.

When it is determined that the first electrode 201 and the second electrode 202 are in contact with the external object, the wearable electronic device 200 may output the series of pulse waves. according to the pulse information. The pulse waves may be output through at least one of the first electrode 201 or the second electrode 202 in operation 404. In other words, the wearable electronic device 200 according to one embodiment may apply the series of pulse waves to the external object. The pulses waves may temporarily create a micro-channel in a surface of a cell membrane of the user's skin. When the micro-channel is created, the mobility of electrons through the micro-channel may increase, thereby reducing skin resistance. The reduced skin resistance may allow for more accurate measurement of biometric information.

In operation 405, the wearable electronic device 200 according to one embodiment may obtain the biometric information using the first electrode 201 and the second electrode 202.

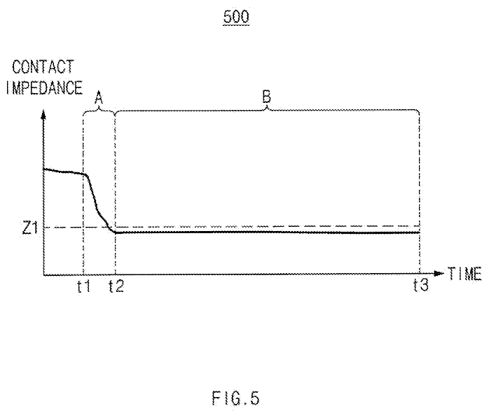

Hereinafter, with reference to FIG. 5, a change in contact impedance that varies according to the application of the series of pulse waves in the wearable electronic device according to one embodiment (e.g., the wearable electronic device 200 in FIG. 2) will be described. FIG. 5 is a graph 500 showing the contact impedance over time of the wearable electronic device 200 according to one embodiment.

Referring to FIG. 5, the wearable electronic device 200 according to one embodiment may output (e.g., operation 404 in FIG. 4) the series of pulse waves through at least one of the first electrode 201 or the second electrode 202 for a pulse output period A that lasts for a first time period t1 to t2. When the series of pulse waves output for the pulse output period A is applied to the user's skin, skin resistance of an area in contact with at least one of the first electrode 201 or the second electrode 202 may decrease. Accordingly, for the pulse output period A, the contact impedance may be gradually lowered.

After the first time period t1 to t2, the wearable electronic device 200 according to one embodiment may acquire (e.g., operation 405 in FIG. 4) a biometric signal for a biometric signal acquisition period B that lasts for a second time period t2 to t3. In this regard, the first time period t1 to t2 and the second time period t2 to t3 might not overlap each other. In one example, the pulse output period A and the biometric signal acquisition period B might not overlap each other. In another example, there may be a specified time period (e.g., a third time period (not shown)) between the pulse output period A and the biometric signal acquisition period B to ensure that output period A and output period B do not overlap.

For the biometric signal acquisition period B, the contact impedance may be smaller than or equal to a target impedance Z1. In other words, the wearable electronic device 200 according to one embodiment may acquire the biometric signal when the contact impedance is smaller than or equal to the target impedance Z1. Because the wearable electronic device 200 according to one embodiment may acquire the biometric signal when the contact impedance has a sufficiently low value (e.g., a value lower than or equal to the target impedance Z1), accuracy of the acquired biometric signal may be improved.

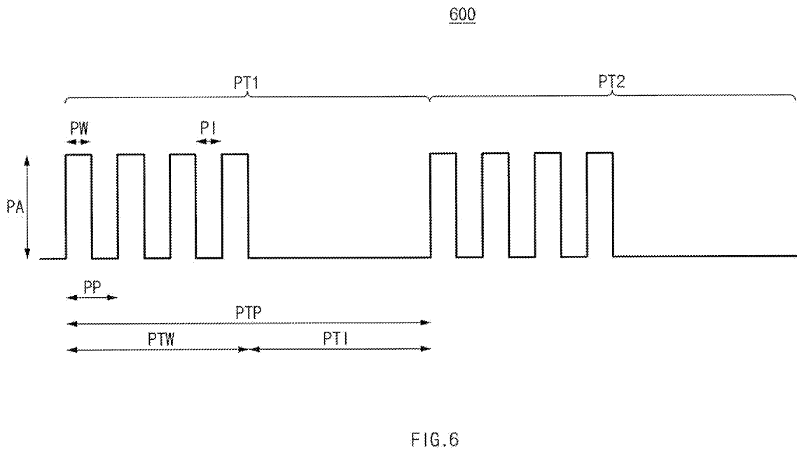

Hereinafter, with reference to FIG. 6, the series of pulse waves output from the wearable electronic device according to one embodiment (e.g., the wearable electronic device 200 in FIG. 2) will be described. FIG. 6 is a waveform diagram 600 of a series of pulse waves output from the wearable electronic device 200 according to one embodiment.

Referring to FIG. 6, the series of pulse waves may include at least one pulse train PT1 and PT2. In this regard, the pulse train PT1 and PT2 may mean one set including consecutive pulses. The parameter of the series of pulse waves included in the pulse information (e.g., the pulse information set in operation 401 of FIG. 4) may include at least one of pulse power (PA), a pulse width (PW), a pulse interval (PI), a pulse period (PP), a pulse train width (PTW), a pulse train interval (PTI), a pulse train period (PTP), the number of pulses included in each of pulse trains PT1 and PT2, a duty cycle (PTW/PTP), a pulse duty cycle (PW/PP), and/or a pulse shape.

The waveform diagram of the series of pulse waves as shown in FIG. 6 is illustrative. The parameter of the series of pulse waves output from the wearable electronic device 200 according to one embodiment is not limited to that as shown in FIG. 6. In one example, FIG. 6 shows that the pulse wave is a square wave. However, the pulse shape is not limited thereto.

In certain embodiments, the wearable electronic device 200 can apply the series of pulses until the impedance is below a specified or predetermined value. Accordingly, the wearable electronic device 200 can include a module for measuring the contact impedance.

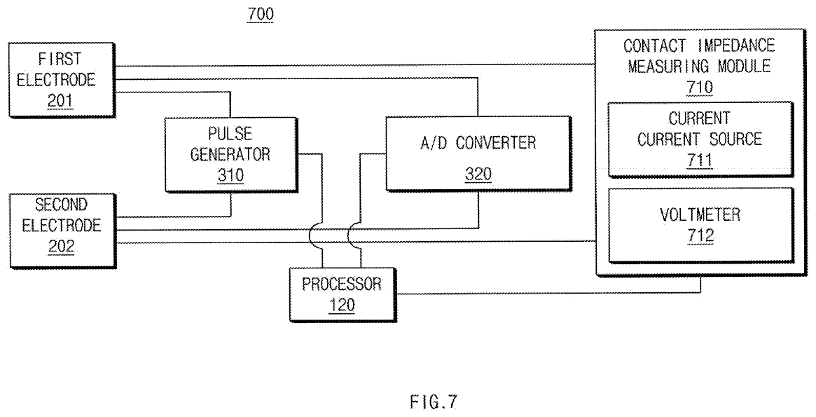

Hereinafter, with reference to FIG. 7, a wearable electronic device according to one embodiment (e.g., the wearable electronic device 200 in FIG. 2) will be described. FIG. 7 is a block diagram 700 of a wearable electronic device 200 according to one embodiment. Hereinafter, a detailed description of the components duplicate with those of FIG. 3 may be referred to the description of FIG. 3.

Referring to FIG. 7, the wearable electronic device 200 according to one embodiment may include the processor 120, the first electrode 201, the second electrode 202, the pulse generator 310, the A/D converter 320, and/or a contact impedance measuring module 710.

The pulse generator 310 may be electrically connected to the first electrode 201 and the second electrode 202 so as to output a series of pulse waves to at least one of the first electrode 201 or the second electrode 202.

The A/D converter 320 may be electrically connected to the first electrode 201 and the second electrode 202 so as to detect biometric information (e.g., electrocardiogram (ECG), bioelectrical impedance analysis (BIA), and/or electrodermal activity (EDA)). The A/D converter 320 may process an electrical signal acquired through the first electrode 201 or the second electrode 202.

The contact impedance measuring module 710 may be electrically connected to the first electrode 201 and/or the second electrode 202 so as to measure the contact impedance of at least one of the first electrode 201 and/or the second electrode 202.

The contact impedance measuring module 710 may include a current source 711 and a voltmeter 712. The current source 711 may apply a predetermine amount of current to at least one of the first electrode 201 or the second electrode 202. The voltmeter 712 may measure voltage across the first electrode 201 and the second electrode 202, when the predetermined amount of current is applied. The contact impedance measuring module 710 may be configured to measure the contact impedance of at least one of the first electrode 201 or the second electrode 202, based on a current value applied by the current source 711 and a voltage value measured by the voltmeter 712. In certain embodiments, the contact impedance measuring module 710 can determine the impedance to be the voltage values measured by the voltmeter 712 divided by the predetermined amount of current. In certain embodiments, the current source 710 and the voltmeter 712 may be connected to a circuit that determine the contact impedance. In another embodiments, the current source 710 and the voltmeter 712 may be connected to the processor 120 and the processor 120 determines the contact impedance.

The processor 120 may be electrically or operatively coupled to or connected to other components (e.g., the pulse generator 310, the A/D converter 320 and the contact impedance measuring module 710) of the wearable electronic device 200.

The processor 120 may determine whether the first electrode 201 and the second electrode 202 are in contact with the external object (e.g., the user's skin). In one example, the wearable electronic device 200 according to one embodiment may determine whether the first electrode 201 and/or the second electrode 202 is in contact with the external object.

Contact can be determined in a variety of ways. In one example, contact may be determined using the first electrode 201 and/or the second electrode 202. In another example, the wearable electronic device 200 according to one embodiment may determine whether the second electrode 202 is in contact with the external object using the optical sensor (221 in FIG. 2). In another example, the processor 120 may periodically activate the contact impedance measuring module 710 to measure the contact impedance of at least one of the first electrode 201 or the second electrode 202 and then may determine whether the first electrode 201 and the second electrode 202 are in contact with the external object (e.g., the user's skin), based on the measured contact impedance. In still another example, the processor 120 may activate the contact impedance measuring module 710 based on a biometric information measuring request and then determine whether the first electrode 201 and the second electrode 202 are in contact with the external object based on the measured contact impedance.

The processor 120 may set the pulse information about the series of pulse waves. The pulse information about the series of pulse waves may include at least one parameter about the series of pulse waves.

The processor 120 may set the target impedance. The wearable electronic device 200 according to one embodiment may measure the biometric information when the contact impedance is lower than the target impedance. According to one embodiment, the processor 120 may set the target impedance using input impedance of the differential amplification circuit included in the A/D converter 320. In one embodiment, the target impedance may be smaller than the input impedance of the differential amplification circuit. In one example, the processor 120 may set a value in a range of 1/1000 to 1/100 of the input impedance of the differential amplification circuit as the target impedance. In other words, the target impedance may be in a range of the input impedance/1000 to the input impedance/100. Therefore, when measuring the biometric signal, the contact impedance may have a smaller value than the input impedance, and decay in the biometric signal due to the contact impedance and the input impedance may be minimized.

The processor 120 may be configured to output the series of pulse waves according to specified pulse information through at least one of the first electrode 201 or the second electrode 202 using the pulse generator 310 in a state where the first electrode 201 and the second electrode 202 are in contact with the external object (e.g., the user's skin).

The processor 120 may obtain the contact impedance measured using the contact impedance measuring module 710. The processor 120 may compare the measured contact impedance with the target impedance.

The processor 120 may be configured to output the series of pulse waves using the pulse generator 310 when the contact impedance exceeds the target impedance. The processor 120 may be configured to repeat the operation of outputting the series of pulse waves using the pulse generator 310 and the operation of measuring the contact impedance using the contact impedance measuring module 710 until the contact impedance has a value equal to or smaller than the target impedance.

The processor 120 may obtain the biometric information using the A/D converter 320 when the contact impedance is equal to or smaller than the target impedance.

Although only the first electrode 201 and the second electrode 202 are shown in FIG. 7, the number of electrodes is not limited thereto. The wearable electronic device 200 according to one embodiment may include three or more electrodes. In one example, the wearable electronic device 200 according to one embodiment may further include the third electrode (203 in FIG. 2) electrically connected to the pulse generator 310, the A/D converter 320 and the contact impedance measuring module 710.

In certain embodiments, the wearable electronic device 200 can wait until the electrodes 201, 202 make contact with an external object. After making contact with the external object, the wearable electronic device 200 outputs a series of pulse waves until the contact impedance is below a target impedance. When the contact impedance is below the contact impedance, the wearable electronic device 200 obtained biometric information.

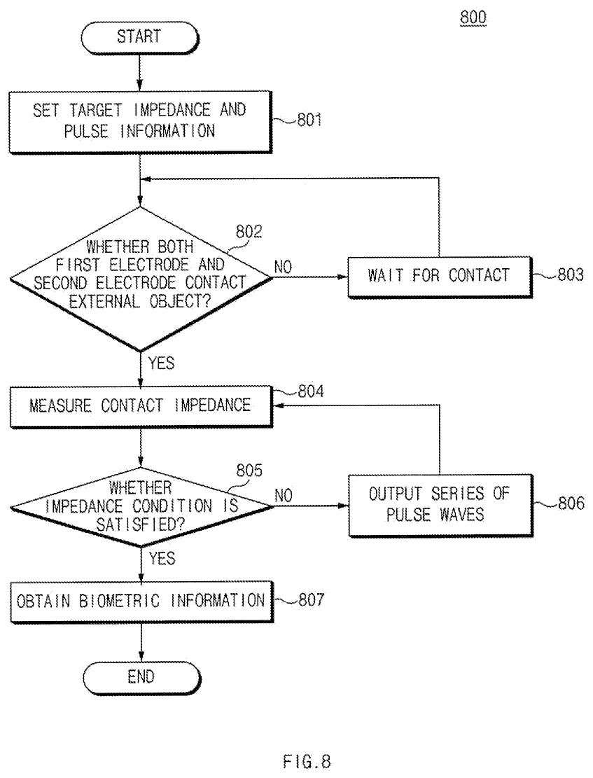

Hereinafter, with reference to FIG. 8, an operation of the wearable electronic device according to one embodiment (e.g., the wearable electronic device 200 in FIG. 2) will be described. FIG. 8 is an operation flowchart 800 of the wearable electronic device 200 according to one embodiment. Hereinafter, a detailed description of the components duplicate with FIG. 4 may be referred to the description of FIG. 4.

In operation 801, the wearable electronic device 200 according to one embodiment may set the target impedance and the pulse information. In one embodiment, the target impedance may be smaller than the input impedance of the differential amplification circuit. In one example, the target impedance may be a value in a range of the input impedance/1000 to the input impedance/100. The pulse information may include at least one parameter about the series of pulse waves.

In operation 802, the wearable electronic device 200 according to one embodiment may determine whether both the first electrode (201 in FIG. 3), and the second electrode (202 in FIG. 3) are in contact with the external object (e.g., the user's skin).

When it is determined that any one of the first electrode 201 and the second electrode 202 is not in contact with the external object, the wearable electronic device 200 according to one embodiment may perform a contact waiting operation in operation 803. In one example, the wearable electronic device 200 according to one embodiment may output a re-wearing request message and/or a re-contacting message to the user via a display (e.g., the display 211 in FIG. 2). In another example, the wearable electronic device 200 according to one embodiment may simply wait for a specified time duration. After performing the contact waiting operation (operation 803), the wearable electronic device 200 according to one embodiment may re-determine (operation 802) whether the first electrode 201 and the second electrode 202 are in contact with the external object.

When it is determined that both the first electrode 201 and the second electrode 202 are in contact with the external object, the wearable electronic device 200 according to one embodiment may measure the contact impedance in operation 804.

In operation 805, the wearable electronic device 200 according to one embodiment may determine whether the measured contact impedance satisfies an impedance condition. In one example, the wearable electronic device 200 according to one embodiment may compare the measured contact impedance and the target impedance with each other and may determine whether the measured contact impedance is smaller than or equal to the target impedance.

When the measured contact impedance exceeds the target impedance or does not satisfy the impedance condition, the wearable electronic device 200 according to one embodiment may output the series of pulse waves according to the pulse information through at least one of the first electrode 201 or the second electrode 202 in operation 806. The wearable electronic device 200 according to one embodiment may output the series of pulse waves (in operation 806) and then measure the contact impedance again (in operation 804).

When the measured contact impedance is smaller than or equal to the target impedance (or when the impedance condition is satisfied), the wearable electronic device 200 according to one embodiment may obtain the biometric information using the first electrode 201 and the second electrode 202 in operation 807.

According to certain embodiments, the wearable electronic device 200 may store the pulse information about the series of pulse waves output in operation 806 in a memory (e.g., the memory 130 of FIG. 1). The wearable electronic device 200 according to one embodiment may set pulse information using the stored pulse information when the same user re-measures a biometric signal. The wearable electronic device 200 according to one embodiment may set the pulse information using the pulse information used when the user's biometric signal was previously measured. In one example, the wearable electronic device 200 may use previously performed target impedance and/or pulse information after detecting that the user is wearing the wearable electronic device 200 and when it is determined that the user is maintaining the wearing state thereof. Therefore, when the same user measures a biometric signal a plurality of times, the operation (operation 804) of measuring the contact impedance at each measurement may not be performed, and the biometric signal may be quickly measured.

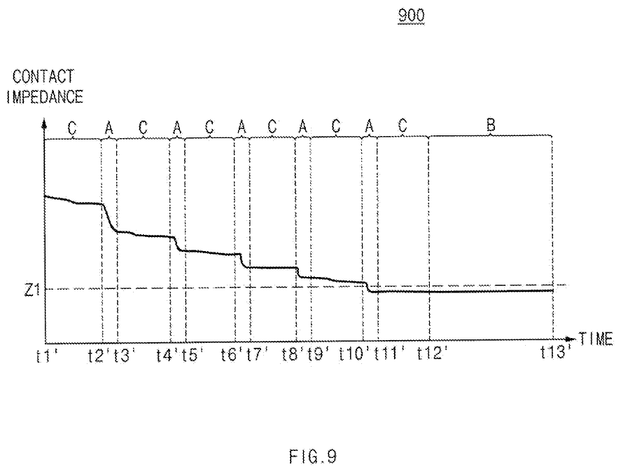

Hereinafter, with reference to FIG. 9, change in contact impedance that changes according to application of a series of pulse waves in a wearable electronic device according to one embodiment (e.g., the wearable electronic device 200 in FIG. 2) will be described. FIG. 9 is a graph 900 showing contact impedance over time of the wearable electronic device 200 according to one embodiment.

Referring to FIG. 9, the wearable electronic device 200 according to one embodiment may measure (e.g., operation 804 in FIG. 8) the contact impedance of at least one of the first electrode 201 and/or the second electrode 202 for the contact impedance measuring period C, and may output (e.g., operation 806 in FIG. 8) the series of pulse waves through at least one of the first electrode 201 and/or the second electrode 202 for the pulse output period A. Further, the wearable electronic device 200 according to one embodiment may acquire (e.g., operation 807 in FIG. 8) a biometric signal for the biometric signal acquisition period B.

The wearable electronic device 200 according to one embodiment may repeatedly perform the operation of the pulse output period A and the operation of the contact impedance measuring period C until it is determined that the contact impedance has a value equal to or smaller than the target impedance Z1. According to one embodiment, the pulse output period A, the biometric signal acquisition period B and the contact impedance measuring period C may not overlap each other. According to one embodiment, there may be a specified time interval between adjacent ones of the pulse output period A, the biometric signal acquisition period B and/or the contact impedance measuring period C.

In one example, the wearable electronic device 200 according to one embodiment may measure the contact impedance for the contact impedance measuring period C that lasts for a first′ time period t1′ to t2′. When the measured contact impedance exceeds the target impedance Z1, after the first′ time period t1′ to t2′, the wearable electronic device 200 according to one embodiment may output the series of pulse waves for the pulse output period A lasting for a second′ time period t2′ to t3′. When the series of pulse waves output for the pulse output period A is applied to the user's skin, the contact impedance may be lowered. After the second′ time period t2′ to t3′, the wearable electronic device 200 according to one embodiment may measure the contact impedance for the contact impedance measuring period C lasting for a third′ time period t3′ to t4′. The wearable electronic device 200 according to one embodiment may repeat the operation of the pulse output period A and the operation of the contact impedance measuring period C until it is determined that the contact impedance is equal to or smaller than the target impedance Z1. In one example, the wearable electronic device 200 according to one embodiment may output the series of pulse waves for a fourth′ time period t4′ to t5′, a sixth′ time period t6′ to t7′, an eighth ‘ time period t8’ to t9′ and a tenth′ time period t10′ to t11′, and may measure the contact impedance for a fifth′ time period t5′ to t6′, a seventh′ time period t7′ to t8′, a ninth′ time period t9′ to t10′ and an eleventh′ time period t11′ to t12′. When it is determined that the measured contact impedance is equal to or smaller than the target impedance Z1, the wearable electronic device 200 according to one embodiment may acquire a biometric signal for the biometric signal acquisition period B that lasts for a twelfth′ time period t12′ to t13′.

All of the series of pulse waves output for a plurality of pulse output periods A may be identical with each other. However, depending on an embodiment, the series of pulse waves output for some of the plurality of pulse output periods A may be different from the series of pulse waves output for the other of the plurality of the pulse output periods A. In one example, at least some of first pulse information of the series of pulse waves output for the pulse output period A for the second′ time period t2′ to t3′ may be different from at least some of second pulse information of the series of pulse waves output for the pulse output period A for the fourth′ time period t4′ to t5′. A parameter of the second pulse information may be set based on a parameter of the first pulse information and the contact impedance measured for the contact impedance measuring period C for the third ‘time period t3’ to t4′. Alternatively, the second pulse information may be set together when the first pulse information is set. In one embodiment, a parameter included in the pulse information of the series of pulse waves may include at least one of pulse power (PA), a pulse width (PW), a pulse interval (PI), a pulse period (PP), a pulse train width (PTW), a pulse train interval (PTI), a pulse train period (PTP), the number of pulses included in each pulse train PT1 and PT2, a duty cycle (PTW/PTP), a pulse duty cycle (PW/PP), and/or a pulse shape

A time interval between adjacent ones of the plurality of pulse output periods A and/or a time interval between adjacent ones of the plurality of impedance measuring period C may be different from each other. In one example, a duration of the second′ time period t2′ to t3′ for which the series of pulse waves is output and a duration of the fourth′ time period t4′ to t5′ for which the series of pulse waves is output may be different from each other. A time duration for which the series of pulse waves is output may be determined based on an amount of change in the contact impedance.

The wearable electronic device 200 according to one embodiment may measure the contact impedance for the contact impedance measuring period C and determine whether the contact impedance has a value smaller than or equal to the target impedance Z1, based on the measured contact impedance. The wearable electronic device 200 according to one embodiment may change the parameter of the pulse information and/or the pulse output period A based on the measured contact impedance, thereby precisely controlling the contact impedance, such that the accuracy of the biometric signal may be improved.

The time period and number of repetitions of the pulse output period A, the biometric signal acquisition period B and the contact impedance measuring period C as shown in FIG. 9 are merely examples. The wearable electronic device 200 according to one embodiment is not limited to the time period and the number of repetitions of each of the periods as shown in FIG. 9.

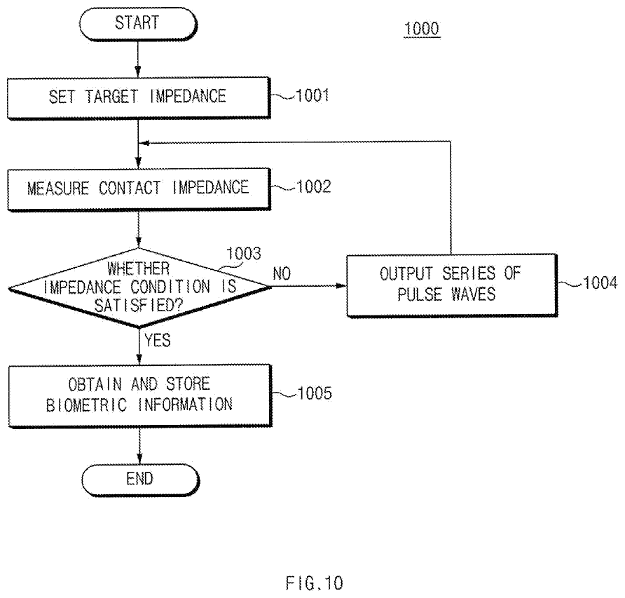

Hereinafter, with reference to FIG. 10, an operation of a wearable electronic device (e.g., the wearable electronic device 200 in FIG. 2) according to one embodiment will be described. FIG. 10 is an operation flowchart 1000 of the wearable electronic device 200 according to one embodiment. Specifically, FIG. 10 shows the operation flowchart 1000 that may be performed when the wearable electronic device 200 according to one embodiment sets pulse information (e.g., in operation 401 in FIG. 4).

In operation 1001, the wearable electronic device 200 according to one embodiment may set the target impedance. In one embodiment, the target impedance may be smaller than the input impedance of the differential amplification circuit. In one example, the target impedance may be a value in a range of the input impedance/1000 to the input impedance/100.

In operation 1002, the wearable electronic device 200 according to one embodiment may measure the contact impedance.

In operation 1003, the wearable electronic device 200 according to one embodiment may determine whether the measured contact impedance satisfies the impedance condition. In one example, the wearable electronic device 200 according to one embodiment may determine whether the measured contact impedance is smaller than or equal to the target impedance, based on a comparing result of the measured contact impedance and the target impedance.

When the measured contact impedance exceeds the target impedance or does not satisfy the impedance condition, the wearable electronic device 200 according to one embodiment may output the series of pulse waves according to the parameter of pulse information through at least one of the first electrode 201 and the second electrode 202 in operation 1004. After outputting the series of pulse waves, the wearable electronic device 200 according to one embodiment may re-measure the contact impedance in operation 1002.

When the measured contact impedance is smaller than or equal to the target impedance (or when the impedance condition is satisfied), the wearable electronic device 200 according to one embodiment may acquire the pulse information in operation 1005. The wearable electronic device 200 according to one embodiment may acquire the pulse information based on parameter information of the series of pulse waves output until the measured contact impedance becomes smaller than or equal to the target impedance. In one example, when the wearable electronic device 200 performs operation 1004 of outputting the series of pulse waves having a first pulse train width (PTW in FIG. 6) a total of n times and then the contact impedance becomes smaller than or equal to the target impedance, (the first pulse train width*n) may be set as a final pulse train width. The wearable electronic device 200 according to one embodiment may store the acquired pulse information in a memory (e.g., the memory 130 in FIG. 1).

The wearable electronic device 200 according to one embodiment may store state information together with the pulse information in the memory. The state information may be information that can be measured in the wearable electronic device 200 in the operation of setting the pulse information. In one example, the state information may include at least one of a temperature, indoor/outdoor information, an atmospheric pressure, intensity of illumination information, movement information using a geomagnetic sensor, position information, or user information.

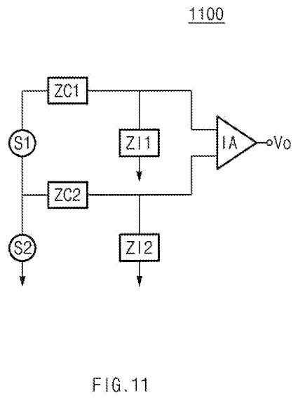

Hereinafter, with reference to FIG. 11, an A/D converter (e.g., the A/D converter 320 of FIG. 3) included in the wearable electronic device 200 according to one embodiment will be described. FIG. 11 is a circuit diagram 1100 showing the A/D converter of the wearable electronic device 200 according to one embodiment.

Referring to FIG. 11, the A/D converter 320 of the wearable electronic device 200 according to one embodiment may include an instrumentation amplifier (IA), a first input impedance ZI1, and a second input impedance ZI2. An output signal Vo of the instrumentation amplifier IA may indicate an electrocardiogram (ECG).

In FIG. 11, a first contact impedance ZC1 may indicate contact impedance generated between the first electrode (e.g., the first electrode 201 in FIG. 3) and the external object. A second contact impedance ZC2 may indicate contact impedance generated between the second electrode (e.g., the second electrode 202 of FIG. 3) and the external object. Further, a first voltage signal Si may indicate a differential input signal, and a second voltage signal S2 may indicate a common input signal.

The instrumentation amplifier IA may reject a common input to an input terminal and amplify a differential input to output the output signal Vo. In this regard, the common input corresponding to an interfering signal may be rejected such that the common gain may be reduced and thus the output signal Vo may include an accurate biometric signal. In one example, as the common mode rejection ratio (CMRR) indicating a ratio of the common gain and the differential gain is increased, quality of the biometric signal may be improved.

Referring to FIG. 11, as a difference between the first contact impedance ZC1 and the second contact impedance ZC2 is greater, the common mode rejection ratio (CMRR) may be reduced. In one example, as the difference between the first contact impedance ZC1 and the second contact impedance ZC2 increases, the common input signal is not rejected, and thus the biometric signal quality may be degraded.

The wearable electronic device 200 according to one embodiment may output the series of pulse waves through an electrode having relatively larger contact impedance among the first electrode 201 or the second electrode 202 to lower the contact impedance. The wearable electronic device 200 according to one embodiment may reduce the difference between the first contact impedance ZC1 and the second contact impedance ZC2 to minimize noise such that the biometric signal quality may be improved.

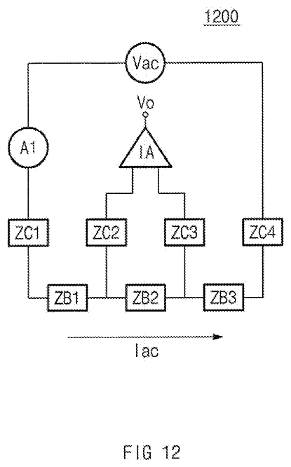

Hereinafter, with reference to FIG. 12, a description will be made of the A/D converter 320 included in the wearable electronic device 200 according to one embodiment. FIG. 12 is a circuit diagram 1200 showing the A/D converter 320 of the wearable electronic device 200 according to one embodiment.

Referring to FIG. 12, the A/D converter 320 of the wearable electronic device 200 according to one embodiment may include the instrumentation amplifier IA and a current source A1. The wearable electronic device 200 according to one embodiment may measure biometric impedances ZB1, ZB2, and/or ZB3 using bioelectrical impedance analysis (BIA). The wearable electronic device 200 according to one embodiment may measure the biometric impedance based on input current Iac applied from the current source A1 and the output signal Vo of the instrumentation amplifier IA.

In FIG. 12, the wearable electronic device 200 according to one embodiment may include four electrodes, and each of contact impedances ZC1, ZC2, ZC3, and/or ZC4 may be generated between each of the electrodes and the external object.

When the contact impedances ZC1, ZC2, ZC3, and/or ZC4 are increased, voltage Vac applied to the living body may be increased, and a maximum value of the input current Iac may be limited.

The wearable electronic device 200 according to one embodiment may output the series of pulse waves through the electrode to lower the contact impedances ZC1, ZC2, ZC3, and/or ZC4. Therefore, the wearable electronic device 200 according to one embodiment may apply a larger input current Iac, so that a signal-to-noise ratio (SNR) may be increased.

A wearable electronic device according to certain embodiments (e.g., the wearable electronic device 200 in FIG. 2) may include a housing (e.g., the housing 230 in FIG. 2), a first electrode (e.g., the first electrode 201) and a second electrode (e.g. the second electrode 201 in FIG. 2) disposed on the housing 230, an A/D converter (e.g., the A/D converter 320 in FIG. 3) connected to the first electrode 201 and the second electrode 202, a pulse generator (e.g., the pulse generator 310 in FIG. 3) connected to the first electrode 201 and the second electrode 202, and a processor (e.g., the processor 120 in FIG. 1) operatively connected to the A/D converter 320 and the pulse generator 310, wherein the processor 120 may be configured to control the pulse generator to output a series of pulse waves to the first electrode 201 when an external object is in contact with the first electrode 201, and to control the A/D converter 320 to obtain biometric information from the first electrode and second electrode in response to outputting the series of pulse waves.

According to certain embodiments, the processor 120 may set pulse information including at least one parameter for the series of pulse waves.

According to certain embodiments, the at least one parameter may include at least one of pulse power, amplitude, a pulse width, a pulse interval, a pulse period, a pulse train width, a pulse train interval, a pulse train period, the number of pulses included in each pulse train, a duty cycle, a pulse duty cycle, or a pulse shape.

According to certain embodiments, the wearable electronic device 200 may further include a contact impedance measuring module (e.g., the contact impedance measuring module 710 in FIG. 7) connected to the first electrode 201 and the second electrode 202, and configured to measure contact impedance of at least one of the first electrode 201 or the second electrode 202.

According to certain embodiments, the contact impedance measuring module 710 may include a current source (e.g., the current source 711 of FIG. 7) for applying a current to at least one of the first electrode 201 or the second electrode 202, and a voltmeter (e.g., the voltmeter 712 of FIG. 7) for measuring voltage between the first electrode 201 and the second electrode 202.

According to certain embodiments, the processor 120 may be configured set a target impedance (e.g., the target impedance Z1 in FIG. 9), and to obtain the biometric information using the A/D converter 320 when the measured contact impedance is smaller than or equal to the target impedance Z1.

According to certain embodiments, the processor 120 may be configured to output a first series of pulse waves to the first electrode 201 for a first time period using the pulse generator 310, and to output a second series of pulse waves to the first electrode 201 using the pulse generator 310 for a second time period non-overlapping the first time period when the contact impedance measured by the contact impedance measuring module 710 exceeds the target impedance Z1.

According to certain embodiments, the at least one parameter includes a first parameter for the first series of pulse waves output for the first time period, and a second parameters for the second series of pulse waves output for the second time period.

According to certain embodiments, the first parameter may be different from the second parameter.

According to certain embodiments, the processor 120 may be configured to set the target impedance Z1, to output a first series of pulse waves to the first electrode 201 for a first time period using the pulse generator 310 and, to output a second series of pulse waves to the first electrode 201 using the pulse generator 310 for a second time period non-overlapping the first time period when the contact impedance measured by the contact impedance measuring module 710 exceeds the target impedance Z1.

According to certain embodiments, the processor 120 may set the pulse information based on a first parameter of the first series of pulse waves a second parameter of the second series of pulse waves when the contact impedance measured by the contact impedance measuring module 710 is smaller than or equal to the target impedance Z1.

According to certain embodiments, the A/D converter 320 may be configured to determine whether the first electrode 201 or the second electrode 202 is in contact with the external object.

According to certain embodiments, the biometric information may be at least one of electrocardiogram (ECG), bioelectrical impedance analysis (BIA), or electrodermal activity (EDA).

The wearable electronic device 200 according to certain embodiments may include a plurality of electrodes (e.g., the first electrode 201, the second electrode 201 and/or the third electrode 203 in FIG. 20), the A/D converter 320 connected to at least one of the plurality of electrodes 201, 202 or 203, the pulse generator 310 connected to at least one of the plurality of electrodes 201, 202 or 203, and the processor 120 operatively connected to the pulse generator 310 and the A/D converter 320, wherein the processor 120 may be configured to output a series of pulse waves through at least one of the plurality of electrodes 201, 202 or 203 using the pulse generator 310 for a pulse output period, and to acquire biometric information using the plurality of electrodes 201, 202 or 203 for a biometric signal acquisition period.

According to certain embodiments, the wearable electronic device 200 further includes the contact impedance measuring module 710 connected to the plurality of electrodes 201, 202 or 203, wherein the processor 120 may be configured to measure contact impedance of at least one of the plurality of electrodes 201, 202 or 203 using the contact impedance measuring module 710 for a contact impedance measuring period.

According to certain embodiments, the processor 120 may be configured to set the target impedance Z1, and to perform an operation (e.g., operation 807 of FIG. 8) of the biometric signal acquisition period when the contact impedance measured by the contact impedance measuring module 710 is smaller than or equal to the target impedance Z1.

According to certain embodiments, when the contact impedance measured by the contact impedance measuring module 710 exceeds the target impedance Z1, the processor 120 may be configured to perform an operation (e.g., operation 806 in FIG. 8) of the pulse output period.

According to certain embodiments, the processor 120 may set pulse information including at least one parameter about the series of pulse waves.

According to certain embodiments, the parameter may include at least one of pulse power, a pulse width, a pulse interval, a pulse period, a pulse train width, a pulse train interval, a pulse train period, a number of pulses included in each pulse train, a duty cycle, a pulse duty cycle, or a pulse shape.

According to certain embodiments, the A/D converter 320 may be configured to determine whether the plurality of electrodes 201, 202 or 203 are in contact with the external object.

According to certain embodiments, a method comprises: outputting a series of pulse waves to a first electrode when an external object is in contact with the first electrode; and obtaining biometric information by an A/D converter from the first electrode and a second electrode in response to outputting the series of pulse waves.

According to certain embodiments, the method further comprises setting at least one parameter for the series of pulse waves.

According to certain embodiments, the at least one parameter includes at least one of pulse power, amplitude, a pulse width, a pulse interval, a pulse period, a pulse train width, a pulse train interval, a pulse train period, a number of pulses included in each pulse train, a duty cycle, a pulse duty cycle, or a pulse shape.

According to certain embodiments, the method further comprises measuring contact impedance of at least one of the first electrode or the second electrode.

According the certain embodiments, measuring impedance comprises applying a current to at least one of the first electrode or the second electrode; and measuring a voltage between the first electrode and the second electrode.

The electronic device according to certain embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that certain embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B”, “at least one of A and B”, “at least one of A or B”, “A, B, or C”, “at least one of A, B, and C”, and “at least one of A, B, or C” may include any one of, or all possible combinations of the items enumerated together in a corresponding one of the phrases. As used herein, such terms as “1st” and “2nd”, or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with”, “coupled to”, “connected with”, or “connected to” another element (e.g., a second element), it means that the element may be coupled with the other element directly (e.g., wiredly), wirelessly, or via a third element.

As used herein, the term “module” may include a unit implemented in hardware, software, or firmware, and may interchangeably be used with other terms, for example, “logic”, “logic block”, “part”, or “circuitry”. A module may be a single integral component, or a minimum unit or part thereof, adapted to perform one or more functions. For example, according to an embodiment, the module may be implemented in a form of an application-specific integrated circuit (ASIC).

Certain embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to certain embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to certain embodiments, each component (e.g., a module or a program) of the above-described components may include a single entity or multiple entities. According to certain embodiments, one or more of the above-described components may be omitted, or one or more other components may be added. Alternatively or additionally, a plurality of components (e.g., modules or programs) may be integrated into a single component. In such a case, according to certain embodiments, the integrated component may still perform one or more functions of each of the plurality of components in the same or similar manner as they are performed by a corresponding one of the plurality of components before the integration. According to certain embodiments, operations performed by the module, the program, or another component may be carried out sequentially, in parallel, repeatedly, or heuristically, or one or more of the operations may be executed in a different order or omitted, or one or more other operations may be added.

What is claimed is:

1. A wearable electronic device comprising:
a housing;
a first electrode and a second electrode disposed on the housing;
an A/D converter connected to the first electrode and the second electrode;
a pulse generator connected to the first electrode and the second electrode; and
a processor operatively connected to the A/D converter and the pulse generator;
wherein the processor is configured to:
control the pulse generator to output a series of pulse waves to the first electrode when an external object is in contact with the first electrode; and
controlling the A/D converter to obtain biometric information from the first electrode and second electrode in response to outputting the series of pulse waves.

2. The wearable electronic device of claim 1, wherein the processor sets at least one parameter for the series of pulse waves.

3. The wearable electronic device of claim 2, wherein the at least one parameter includes at least one of pulse power, amplitude, a pulse width, a pulse interval, a pulse period, a pulse train width, a pulse train interval, a pulse train period, a number of pulses included in each pulse train, a duty cycle, a pulse duty cycle, or a pulse shape.

4. The wearable electronic device of claim 2, further comprising a contact impedance measuring module connected to the first electrode and the second electrode, and configured to measure contact impedance of at least one of the first electrode or the second electrode.

5. The wearable electronic device of claim 4, wherein the contact impedance measuring module includes:
a current source for applying a current to at least one of the first electrode or the second electrode; and
a voltmeter for measuring a voltage between the first electrode and the second electrode.

6. The wearable electronic device of claim 4, wherein the processor is configured to:
set a target impedance; and
when the measured contact impedance is smaller than or equal to the target impedance, obtain the biometric information using the A/D converter.

7. The wearable electronic device of claim 6, wherein the processor is configured to:
output a first series of pulse waves to the first electrode for a first time period using the pulse generator; and
when the contact impedance measured by the contact impedance measuring module exceeds the target impedance, output a second series of pulse waves to the first electrode using the pulse generator for a second time period non-overlapping the first time period.

8. The wearable electronic device of claim 7, wherein the at least one parameter includes:
a first parameter for the first series of pulse waves output for the first time period; and
a second parameter for the second series of pulse waves output for the second time period.

9. The wearable electronic device of claim 8, wherein the first parameter is different from the second parameter.

10. The wearable electronic device of claim 4, wherein the processor is configured to:
set a target impedance;
output a first series of pulse waves to the first electrode for a first time period using the pulse generator; and
when the contact impedance measured by the contact impedance measuring module exceeds the target impedance, output a second series of pulse waves to the first electrode using the pulse generator for a second time period non-overlapping the first time period.

11. The wearable electronic device of claim 10, wherein the processor is configured to:
when the contact impedance measured by the contact impedance measuring module is smaller than or equal to the target impedance, set a first parameter for the first series of the pulse waves and a second parameter for the second series of pulse waves.

12. The wearable electronic device of claim 1, wherein the A/D converter is configured to determine whether the first electrode or the second electrode is in contact with the external object.

13. The wearable electronic device of claim 1, wherein the biometric information is at least one of electrocardiogram (ECG), bioelectrical impedance analysis (BIA), or electrodermal activity (EDA).

14. The wearable electronic device of claim 4, wherein the processor is configured to:
set a target impedance;
repeat an operation of outputting the series of pulse waves to the first electrode and an operation of measuring the contact impedance until the contact impedance measured by the contact impedance measuring module becomes smaller than or equal to the target impedance; and
set parameters of the output series of pulse waves until the measured contact impedance becomes smaller than or equal to the target impedance.

15. The wearable electronic device of claim 6, wherein the A/D converter includes a differential amplification circuit,
wherein the target impedance is smaller than input impedance of the differential amplification circuit.

16. A method comprising:
outputting a series of pulse waves to a first electrode when an external object is in contact with the first electrode; and
obtaining biometric information by an A/D converter from the first electrode and a second electrode in response to outputting the series of pulse waves.

17. The method of claim 16, further comprising setting at least one parameter for the series of pulse waves.

18. The method of claim 17, wherein the at least one parameter includes at least one of pulse power, amplitude, a pulse width, a pulse interval, a pulse period, a pulse train width, a pulse train interval, a pulse train period, a number of pulses included in each pulse train, a duty cycle, a pulse duty cycle, or a pulse shape.

19. The method of claim 17, further comprising measuring contact impedance of at least one of the first electrode or the second electrode.

20. The method of claim 19, wherein measuring impedance further comprises:
applying a current to at least one of the first electrode or the second electrode; and
measuring a voltage between the first electrode and the second electrode.
